(19) United States
(12) Patent Application Publication
(10) Pub. No.: US 2013/0296797 A1
(43) Pub. Date: Nov. 7, 2013

(54) Medical Needle Safety Devices

(71) Applicant: Barry Peter Liversidge, Essex (GB)

(72) Inventor: Barry Peter Liversidge, Essex (GB)

(21) Appl. No.: 13/979,062

(22) PCT Filed: Jan. 11, 2012
(86) PCT No.: PCT/GB2012/050049

§ 371 Date: Jul. 10, 2013

(30) Foreign Application Priority Data
Jan. 12, 2011 (GB) 1100488.4

(51) Int. Cl. A61M 5/32

(52) U.S. Cl. CPC A61M 5/3243
USPC 604/198

(57) ABSTRACT

A safety device 20 for shielding a medical needle 25 comprises a needle mount 28 supporting a medical needle and a needle shielding sleeve 34 arranged coaxially with the mount for sliding movement from an initial needle shielding position to a non-shielding position whereat at least the tip of the needle is exposed beyond the sleeve. Either the sleeve 34 or the mount 28 has both an abutment surface 31 and a sliding surface 29. A flexible connection mounts a radially deformable resilient finger 37 on the other of the sleeve and mount so that a first finger part 46 is configured for radial alignment with the abutment surface 31 where the finger blocks movement of the sleeve or for radial alignment with the sliding surface. A control member 40 is slidable coaxially with the sleeve and is co-operable with a second finger part 47 to the other side of said flexible connection. The control member 40 has a set position at which the first finger part is in radial alignment with the sliding surface so allowing movement of the sleeve from its initial shielding position towards its non-shielding position. This causes deformation of the finger which urges the sleeve back to a shielding position. The movement of the sleeve also causes the control member to be displaced from the set position. This movement of the control member 40 drives the second finger part outwardly which urges the first finger part inwardly and so into radial alignment with the abutment surface following the return of the sleeve to a shielding position. Subsequent movement of the sleeve is blocked by the finger engaging the abutment surface.

This invention concerns safety devices for medical needles and in particular for a medical needle projecting forwardly from the forward end of an injector body. In particular, but not exclusively, this invention relates to a safety device for use with a syringe having a needle permanently affixed thereto, such as a pre-filled single-use syringe having a staked-in needle.

In its preferred aspects, this invention relates to improvements in the safety devices disclosed in my earlier International Patent Publication No. WO 2011/092518 and International Patent Application No. PCT/GB2011/052384, filed respectively on 1 Feb. 2011 and 2 Dec. 2011. These applications will hereinafter be referred to as “said earlier applications” and the entire specifications including the descriptions of the drawings of said earlier applications are incorporated herein by this reference thereto.

The needle safety device of this invention is intended for use with a medical injector supporting a needle used to penetrate a human or animal body, or for other medical uses such as the penetration of a pierceable membrane of an intravenous medication system. In the following, all medical uses of the needle safety device will be described simply as the penetration of a body, even though specific embodiments may be intended for other medical uses.

Throughout this specification the terms “forward” and “forwardly” used in relation to the needle safety device and an injector for use therewith refer to those ends of the components which are approached to a body when a procedure is to be performed, and the direction towards those ends. Conversely, the terms “rearward” and “rearwardly” refer to those ends of the components opposed to the forward ends and the direction away from those forward ends.

In said earlier applications, there are described medical needle safety devices having a needle mount, a needle shielding sleeve movable from an initial shielding position to a non-shielding position and a radially deformable resilient finger provided on one of the sleeve and mount and having an initial substantially undeformed condition. There is a control member having a set position at which sliding movement of the sleeve from its initial position enables engagement of the finger with the control member to deform and lift the finger outwardly, following which continued movement of the sleeve towards its non-shielding position increases the outward deformation of the finger and stores energy therein. On subsequent return of the sleeve to its shielding position under the action of the stored energy, the control member is moved from its set position and the finger returns to its substantially undeformed condition; thereafter the finger blocks movement of the sleeve towards its non-shielding position.

This invention stems from further development of the safety devices of said applications and in particular (but not exclusively) for a pre-filled syringe having a staked-in needle protected by a needle cover until the syringe is to be used. A particular aim of this invention is to provide an improved form of safety device which may be stored indefinitely and which, when required for use, operates reliably to confer passive protection to a needle with which the device is being used.

According to a first aspect of this invention, there is provided A safety device for shielding a medical needle having a sharp tip, which device comprises:
a needle mount for directly or indirectly supporting a medical needle;
a needle shielding sleeve for co-axially surrounding a supported needle and arranged for sliding movement from an initial needle shielding position to a non-shielding position whereat at least the tip of a supported needle is exposed beyond the sleeve;
an abutment surface and a sliding surface provided on one of the sleeve and mount;
a radially deformable resilient finger provided on the other of the sleeve and mount by a flexible connection, a first part of the finger to one side of the connection being configured for radial alignment with the abutment surface or the sliding surface whereby when in radial alignment with the abutment surface movement of the sleeve from its initial needle shielding position is blocked and when in radial alignment with the sliding surface movement of the sleeve from its initial shielding position is permitted while causing deformation of the finger thereby generating a restorative force on the sleeve to urge the sleeve back to a shielding position; and
a control member arranged for coaxial movement with respect to the finger and co-operable with a second part thereof to the other side of said flexible connection, the control member having a set position with respect to the finger at which said first part of the finger is in radial alignment with the sliding surface so allowing movement of the sleeve from its initial shielding position, the control member also having an active position displaced from set position and whereat said second part of the finger is moved outwardly so urging the first part of the finger inwardly into radial alignment with the abutment surface following the return of the sleeve to a shielding position thereby to block subsequent movement of the sleeve away therefrom, the control member being moved to its active position by the use of the device.

In a preferred aspect, the safety device is arranged to have:
a needle mount for directly or indirectly supporting a medical needle;
a needle shielding sleeve for surrounding the needle and arranged coaxially with the mount so that a force applied to the sleeve slides the sleeve from an initial needle shielding position to a non-shielding position whereat at least the tip of the needle is exposed beyond the sleeve;
an abutment surface and a sliding surface provided on one of the sleeve and mount;
a radially deformable resilient finger provided on the other of the sleeve and mount by a flexible connection, a first part of the finger to one side of the connection being configured for radial alignment with the abutment surface or the sliding surface and when in radial alignment with the abutment surface the finger blocks movement of the sleeve from its initial needle shielding position; and
a control member slidably arranged coaxially with the sleeve and mount and co-operable with a second part of the finger to the other side of said flexible connection, the control member having a set position with respect to the sleeve at which said first part of the finger is in radial alignment with the sliding surface so allowing movement of the sleeve from its initial shielding position, and movement of the sleeve towards its non-shielding position causes deformation of the finger thereby generating a restorative force between the finger and the sliding surface that urges the sleeve back to a shielding position, and said movement of the sleeve from the initial shielding position causes the control member to be displaced from the set position;

wherein the movement of the control member from its set position and the return of the sleeve to a shielding position moves said second part of the finger outwardly and by virtue of the flexible connection of the finger also urges the first part of the finger inwardly and so into radial alignment with the abutment surface following the return of the sleeve to a shielding position, to block subsequent movement of the sleeve away therefrom.

In said earlier applications, one end region of the finger is attached to either the sleeve or the mount, but in many preferred embodiments the finger is provided on the sleeve. The other end of the finger engages an abutment surface when the sleeve has been moved from its initial shielding position to a non-shielding position and back to a shielding position, so as to block movement of the sleeve to expose the needle for a second time. A control member allows, or is arranged to move the finger outwardly to allow, the rearward movement of the sleeve (relative to the syringe and needle) and subsequently allows the finger to return to its undeformed condition so as thereafter to block movement of the sleeve.

In the present invention, the finger is provided on either the sleeve or the mount by means of a flexible connection arranged partway between the ends of the finger such that the finger behaves as a first order lever. The control member co-acts with the finger to permit that rearward movement of the sleeve by the finger sliding on the sliding surface and to be deformed thereby, so as to provide a restorative force on the sleeve, and that following the return of the sleeve to a shielding position, the sleeve is then blocked against rearward movement for a second time.

In one preferred embodiment, the finger and the flexible connection are moulded so that in their undeformed condition and with the control member in its set position ready for use, the first part of the finger is in radial alignment with the sliding surface to be permitted to slide therealong. In use of the device, the control member is moved from its set position so that on return of the sleeve to a shielding position, the finger will thereafter block rearward movement of the sleeve for a second time.

In another preferred embodiment, the control member when in its set position holds the first part of the finger in radial alignment with the sliding surface to be permitted to slide therealong but in the course of use of the device the control member moves from its set position so as to hold the finger in a position where the finger will block subsequent rearward movement of the sleeve.

The actions described above in relation to the two preferred embodiments are achieved by the control member having a set (or an initial) position where the safety device is ready to use, but in use of the device, the control member is moved from its set position to act on the second part of the finger (i.e. the end of the finger remote from the end which engages the abutment surface) to block movement of the sleeve. By moving the second part outwardly, the first part of the finger is urged to move inwardly as the finger is in effect a first order lever turning about the flexible connection, so that when the sleeve returns to a shielding position, the sleeve will thereafter be blocked from moving rearwardly.

It is possible to configure a safety device of this invention with the sleeve sliding over the mount or with the sleeve sliding within the mount. In the case of the former, the flexible connection of the finger to the sleeve should be adjacent the forward end thereof such that the rearward end of the finger may engage the abutment surface of the mount to block rearward movement of the sleeve following use of the device. In the case of the latter, the flexible connection of the finger should be to the mount such that the forward end of the finger may engage the abutment surface of the sleeve to block rearward movement of the sleeve following use of the device.

In one embodiment of this invention, the control member when in its set position is disposed adjacent the mount and is arranged to slide forwardly with respect to the sleeve as the sleeve slides rearwardly from its initial shielding position to expose at least the tip of a needle, in the course of performing an injection. On the subsequent return of the sleeve to a shielding position, the control member remains stationary with respect to the sleeve and thus is displaced from its set position and acts on the second part of the finger to bring the first part of the finger into radial alignment with the abutment surface.

In a second embodiment of this invention, the control member when in its set position is disposed at the front end of the sleeve and has a portion which projects beyond that front end, such that in use the control member is moved away from its set position by contact with skin at an injection site as the needle is moved forwardly into that site. In the course of the control member moving rearwardly with respect to the sleeve from its set position, the control member acts on the second part of the finger to bring the first part of the finger into radial alignment with the abutment surface.

The control member in this embodiment may have a recess to accommodate the forward end of the finger when the control member is in its set position. When the control member moves from its set position the forward end of the finger comes out of the recess so as to be acted on by the control member and cause movement of the finger about the flexible connection to bring the first part of the finger into radial alignment with the abutment surface.

The device of this invention is particularly suitable for use with a pre-filled syringe. In one arrangement, the syringe is wholly contained in the mount but with the plunger of the syringe projecting rearwardly therefrom. In this case, the sleeve may slide internally within the mount. Then, the control member may be arranged for movement with respect to the mount, for example by being associated with the plunger. Thus, the control member may be either fixed to or slidable on the plunger, to control operation of the fingers in the course of performing an injection.

Accordingly, another but closely related aspect of this invention provides a safety device of this invention as described above in combination with an injection device comprising a syringe having a medical needle with a sharp tip, a plunger for expelling a contained liquid drug though the needle and a safety device for shielding the needle, wherein the control member is associated with the plunger and is arranged such that movement of the plunger to expel the drug from the syringe displaces the control member from the set position and moves said second part of the finger outwardly so urging the first part of the finger inwardly by virtue of the flexible connection of the finger. In this way, the first part of the finger comes into radial alignment with the abutment surface following the return of the sleeve to a shielding position and the finger then blocks subsequent movement of the sleeve away from that shielding position.

Preferably, in this arrangement, the abutment surface and the sliding surface are provided on the sleeve and the finger is carried by the mount to project forwardly for contacting the abutment surface or the sliding surface on the sleeve.

In any of the arrangements of this invention, the final axial shielding position of the sleeve, when blocked against rearward movement, may be exactly the same as the initial axial shielding position thereof, or may differ slightly from that initial position. It may be advantageous in some embodiments to allow the sleeve to have a small degree of freedom of movement in the axial direction when in its shielding position, to assure proper operation of the safety device. In such a case, the initial position of the sleeve may be at one extreme of that freedom of movement and the final position at the other extreme of that freedom of movement. Despite this possible variation in the initial and final positions of the sleeve, the final position will be substantially the same as the initial position and functionally will be the same, in that in both positions the needle is fully shielded by the sleeve. Thus, it will be appreciated that the initial shielding position of the sleeve and the further shielding position thereof may in fact be the same axial position.

Though the invention has been described above in relation to a safety device having a single finger, it is contemplated that a device will have typically two diametrically opposed fingers or three or more fingers equi-spaced around the device though other numbers of fingers could be employed as may be required.

This invention extends to a safety device of this invention in combination with a medical needle projecting forwardly from a hub, the needle mount having a bore for receiving that hub. The hub may comprise a part of a syringe having a needle permanently affixed thereto and projecting forwardly therefrom whereby the device is mounted to the syringe in order to give passive protection to the needle of the syringe. In its preferred aspects, the syringe comprises a pre-filled single-use syringe having a permanently attached staked-in needle.

By way of example only, several specific embodiments of safety device of this invention will now be described in detail, reference being made to the accompanying drawings in which:

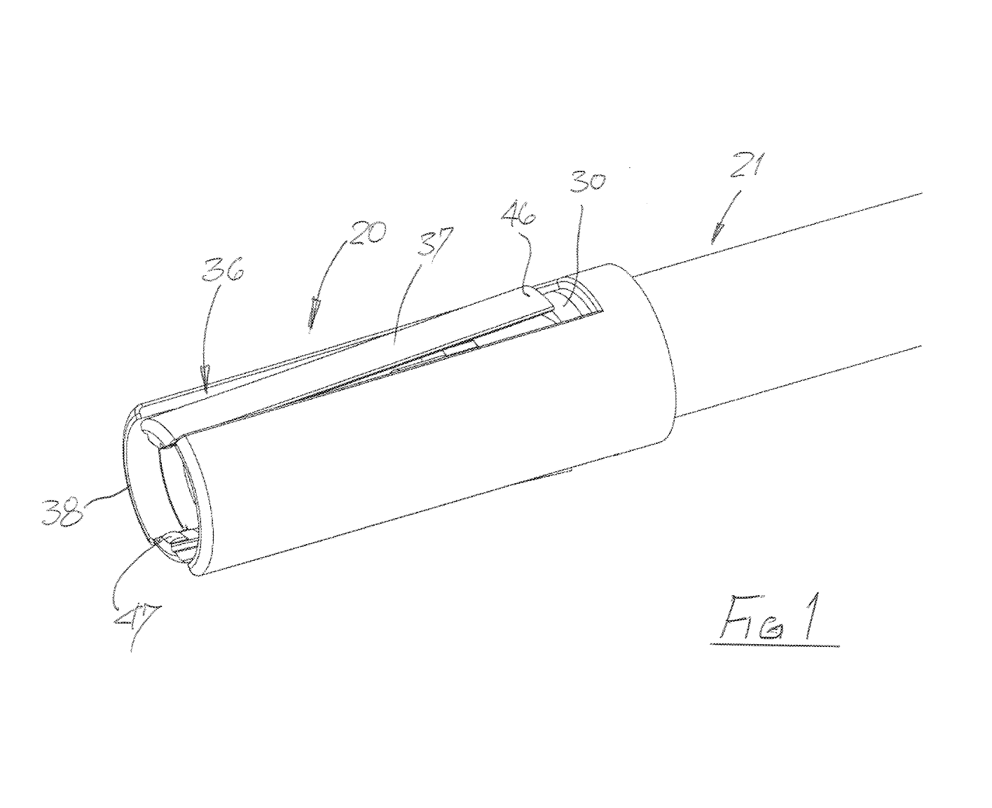

FIG. 1 is an isometric view of the first embodiment in its “set” position ready for use;

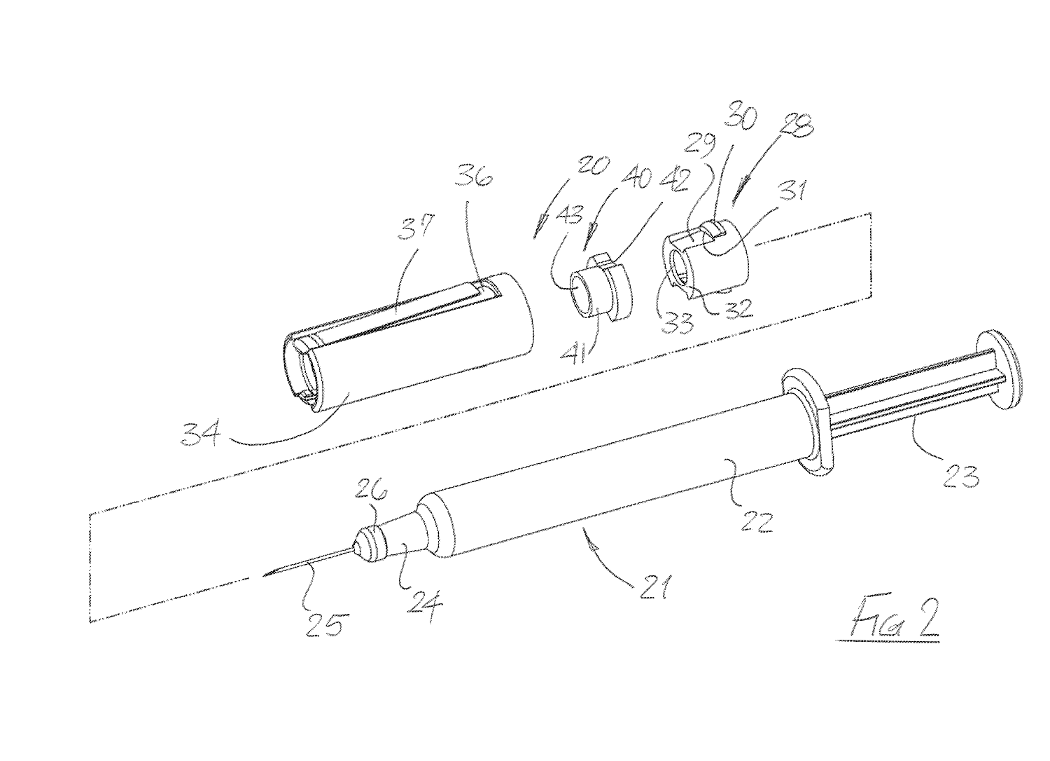

FIG. 2 is an isometric exploded view of the device of FIG. 1;

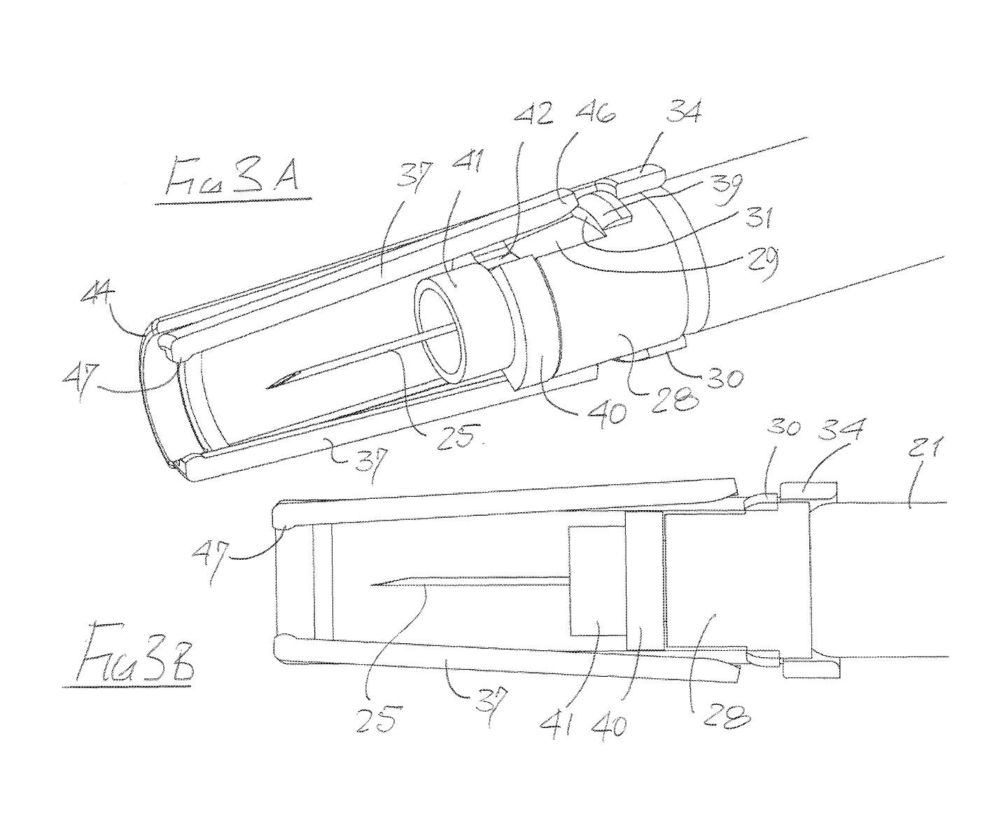

FIGS. 3A and 3B are respectively sectional isometric and axial views of the device of FIG. 1, in its set position;

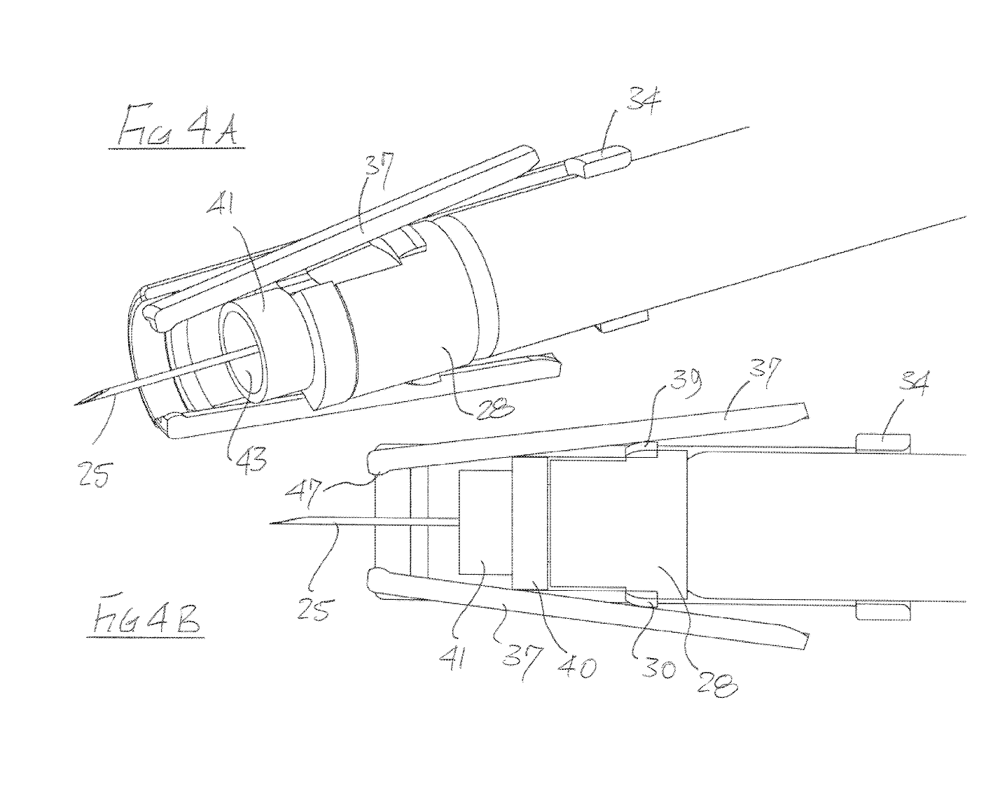

FIGS. 4A and 4B, 5A and 5B, 6A and 6B and 7A and 7B are respectively sectional isometric and axial views of the device of FIG. 1, showing successive steps in the course of use of the device, from its set position of FIGS. 3A and 3B;

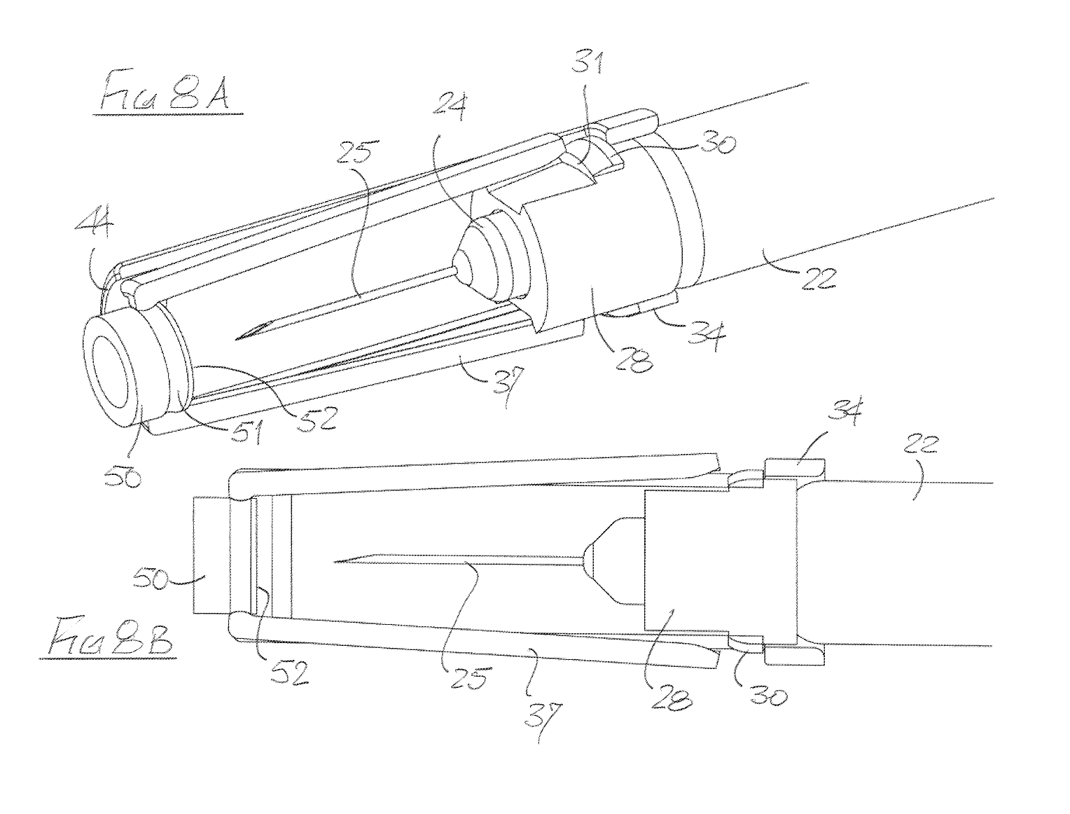

FIGS. 8A and 8B are respectively sectional isometric and axial views of a second embodiment of device of this invention, in its set position;

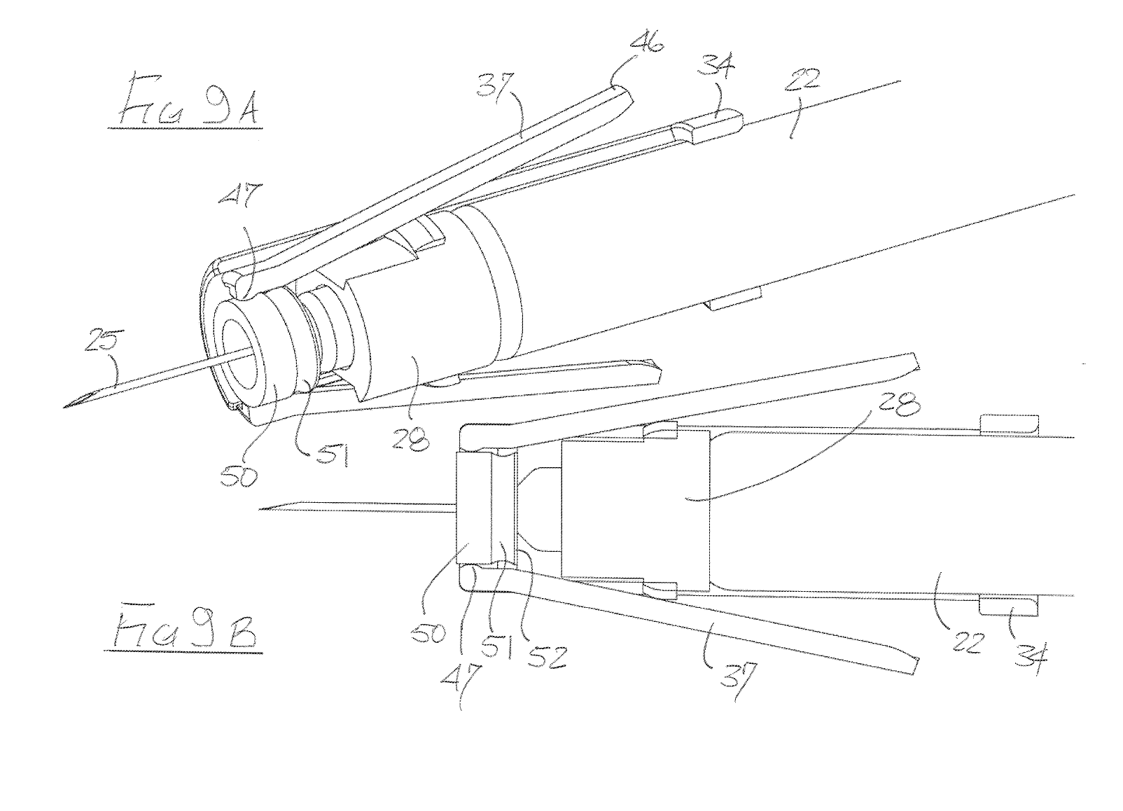

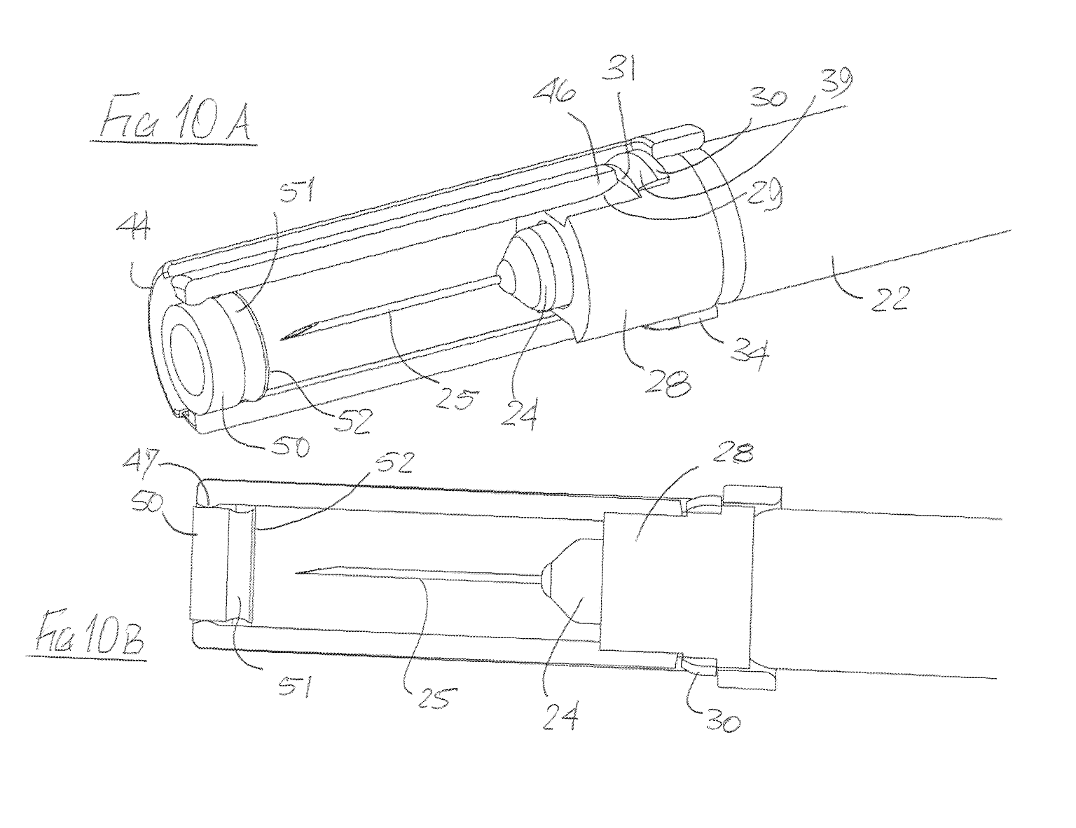

FIGS. 9A and 9B and 10A and 10B are respectively sectional isometric and axial views of the second embodiment, showing successive steps in the course of use of the device, from its set position of FIGS. 8A and 8B;

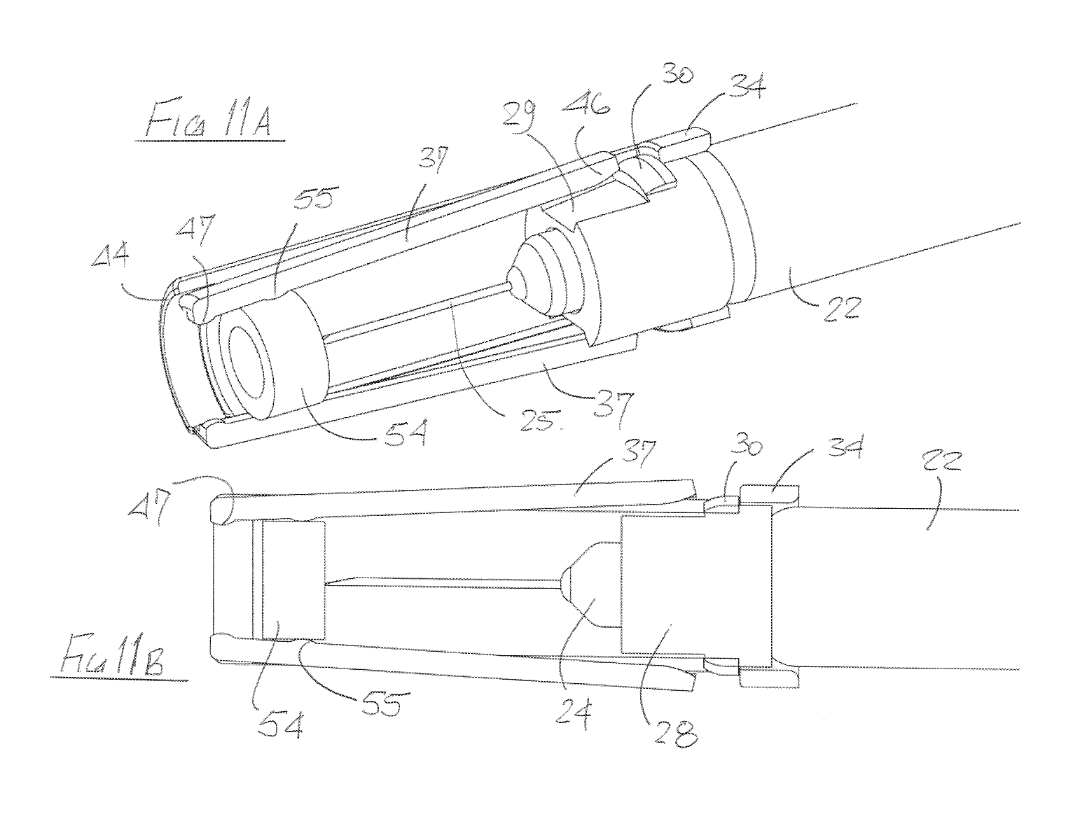

FIGS. 11A and 11B are respectively sectional isometric and axial views of a third embodiment of device of this invention, in its set position;

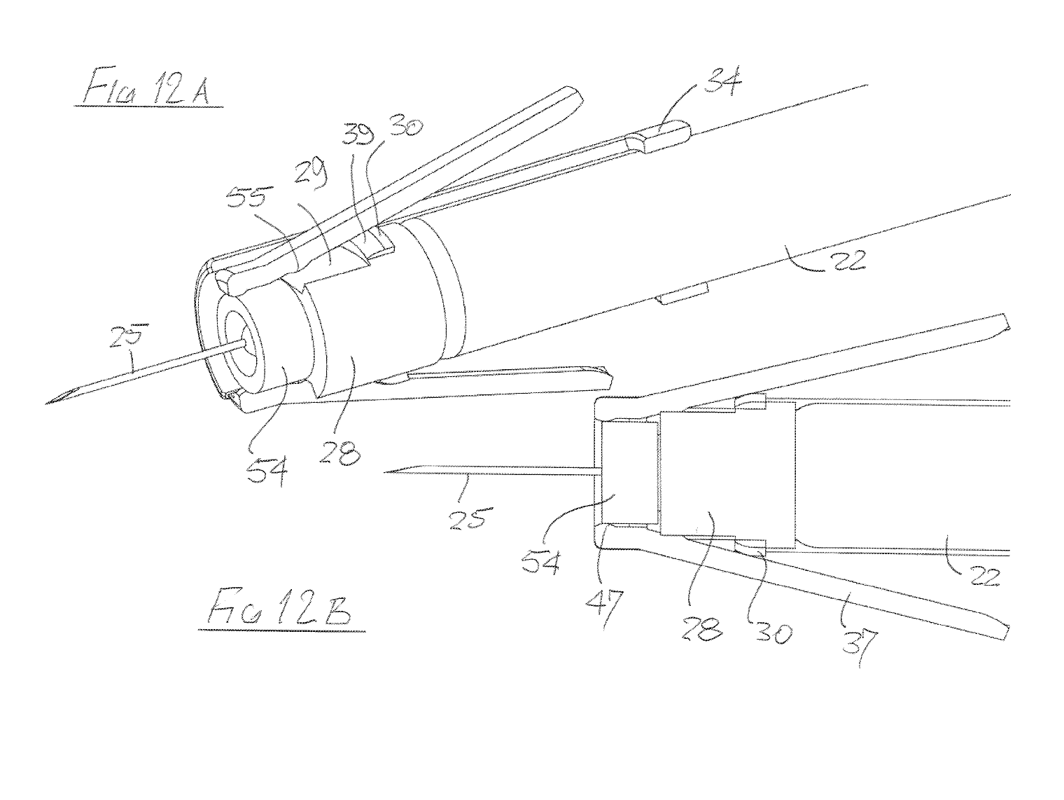

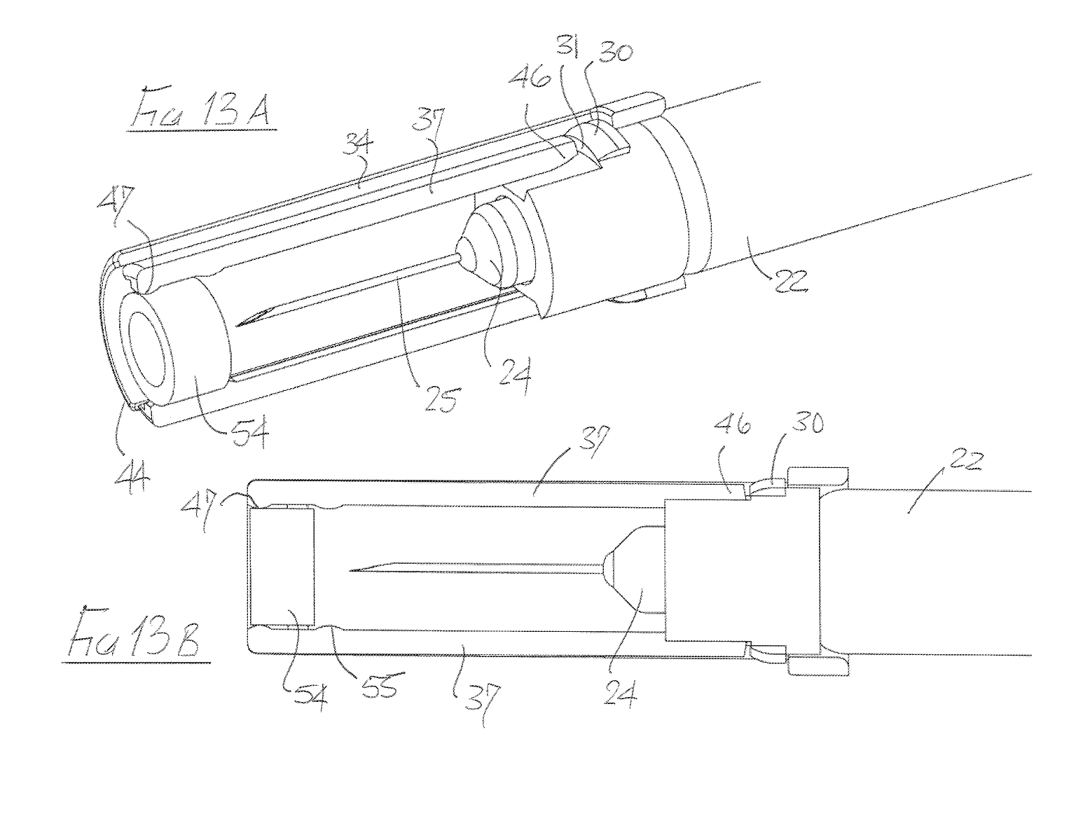

FIGS. 12A and 12B and 13A and 13B are respectively sectional isometric and axial views of the third embodiment, showing successive steps in the course of use of the device, from its set position of FIGS. 11A and 11B;

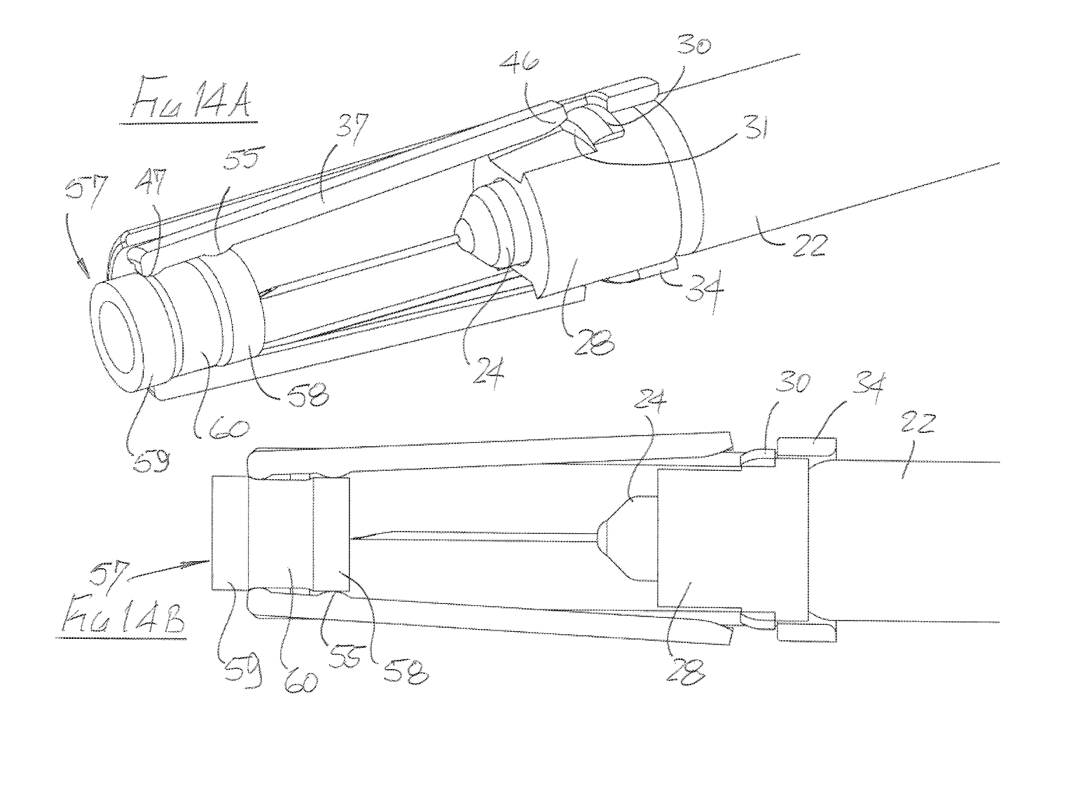

FIGS. 14A and 14B are respectively sectional isometric and axial views of a fourth embodiment of device of this invention, in its set position;

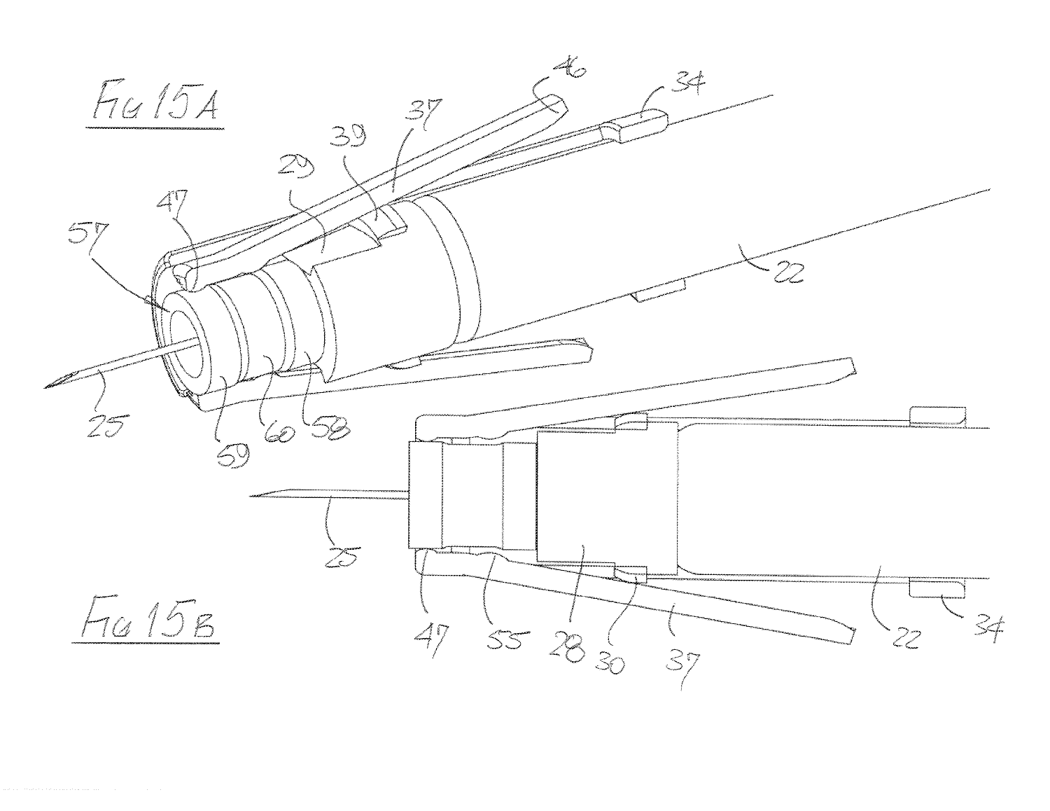

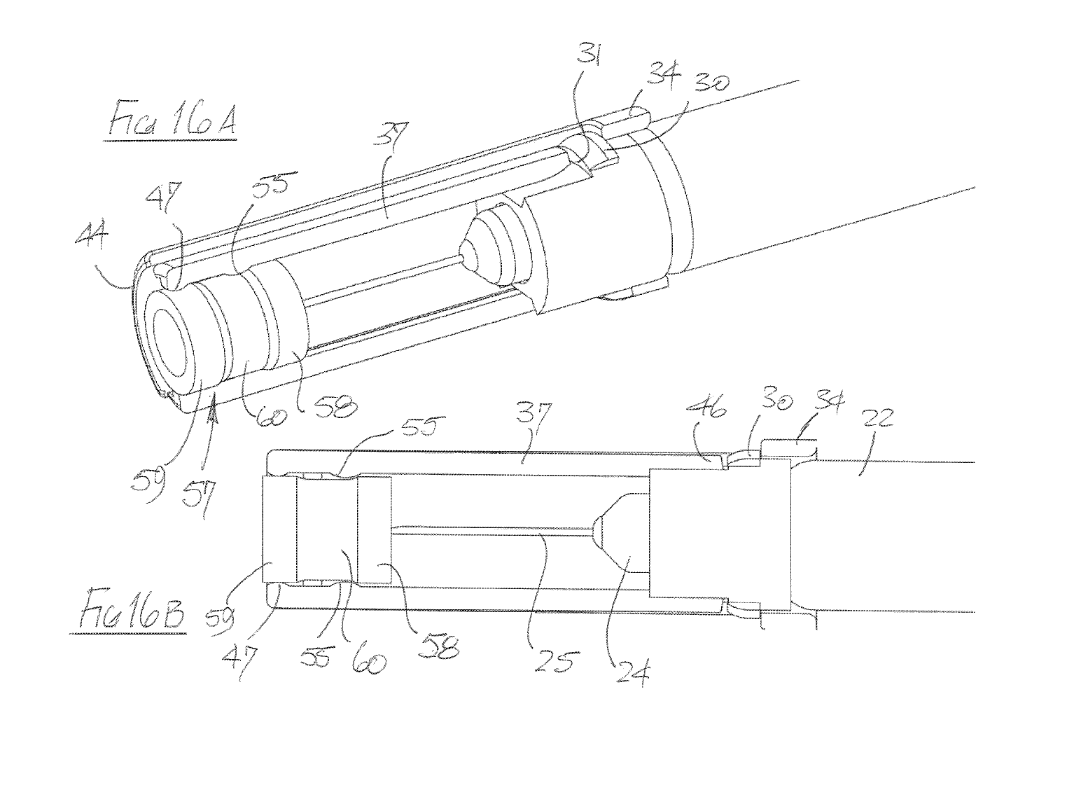

FIGS. 15A and 15B, and 16A and 16B are respectively sectional isometric and axial views of the fourth embodiment, showing successive steps in the course of use of the device, from its set position of FIGS. 14A and 14B;

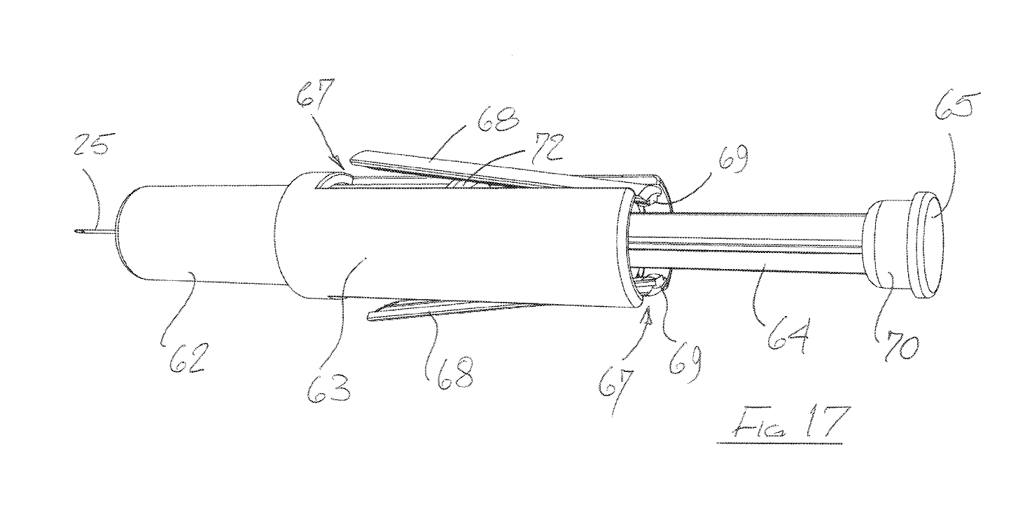

FIG. 17 is an isometric view of a fifth embodiment of this invention, part way through use of the device; and

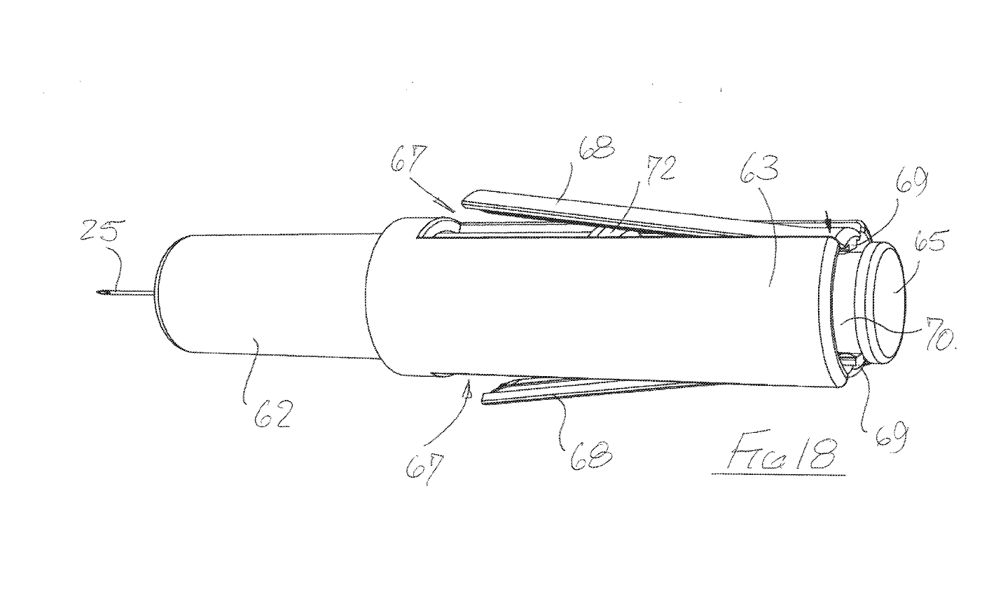

FIG. 18 is a further isometric view of the fifth embodiment but at the completion of the drug dispensing step.

Throughout the following description of the preferred embodiments of safety device of this invention, the same reference characters are used to identify corresponding parts of the various embodiments. Only the first embodiment will be described in complete detail and for a full understanding of the second to fourth embodiments, reference may be made to the description of the first embodiment. Also, where a reference is made to a Figure number without a suffix, that should be understood as referring to each Figure having that number together with a suffix.

The first embodiment of safety device of this invention is shown in FIGS. 1 to 7. The safety device 20 is shown mounted on a conventional medical syringe 21 having a barrel 22 and a plunger 23 (see particularly FIG. 2) supporting a piston (not shown) within the bore of the barrel. At its forward end, the syringe has a nose 24 carrying a staked-in (i.e. permanently attached) hollow needle 25 through which a liquid drug is discharged on depression of the plunger. The forward end of the nose 24 has an annular rib 26 for a purpose to be described below. Typically, the syringe will be pre-filled during manufacture with a liquid drug and then will be stored with a cover over the needle. The safety device may also be fitted to the syringe during manufacture or may be fitted subsequent thereto, for example immediately before the syringe is to be used, though still with the cover over the needle.

The safety device 20 has a generally cylindrical mount 28 the external diameter of which is substantially the same as that of the syringe barrel 22. A pair of diametrically opposed ramp surfaces 29 are formed in the external surface of the mount 28, the rearward end of each ramp surface providing an upstanding lug 30, the forwardly directed face of each lug defining an abutment surface 31. The mount has a front wall 32 in which is formed a hole 33 sized so that the mount may be snap-fitted over the annular rib 26 at the forward end of the syringe nose 24, whereby the mount may be securely carried at the forward end of the syringe, as shown in FIGS. 3 to 10.

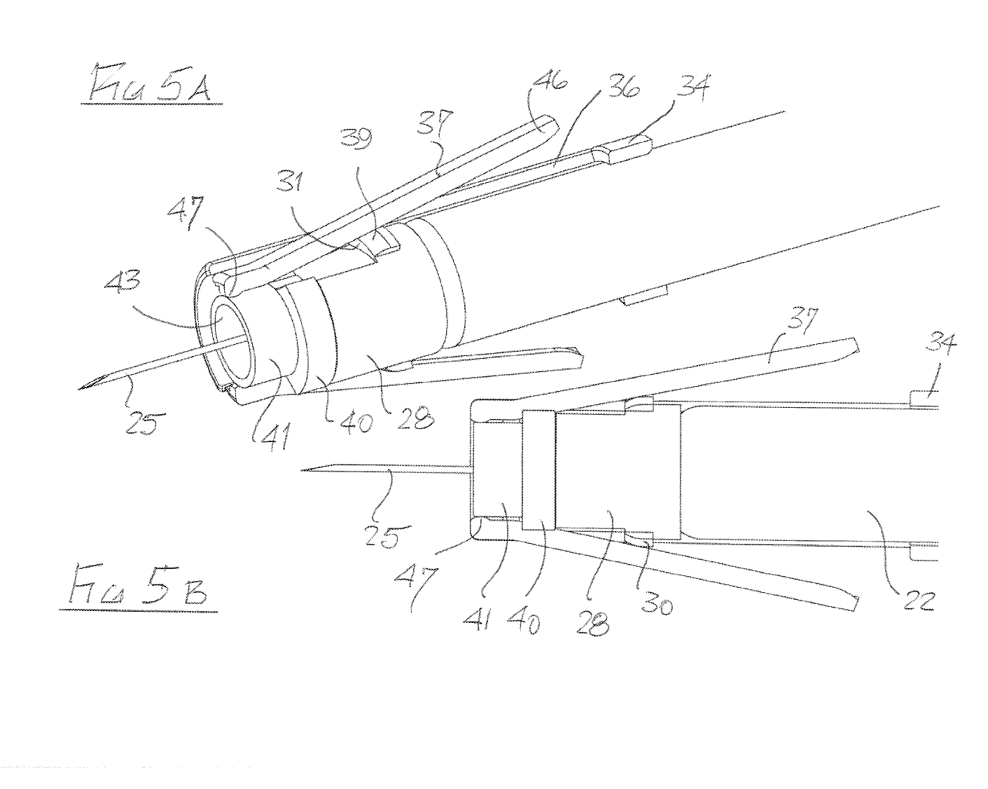

The safety device also includes a sleeve 34 arranged for axial sliding movement with respect to the mount 28 and so also with respect to the syringe 21 and staked-in needle 25. The initial needle-shielding position is shown in FIGS. 1 and 3, and the sleeve may slide rearwardly with respect to the syringe to a non-shielding position shown in FIG. 5, where part of the needle back from its sharp tip is exposed, so that a medical procedure such as an injection may be performed. The sliding movement of the sleeve may occur as a part of that procedure.

The sleeve has a pair of opposed elongate apertures 36 within which are furnished respective fingers 37 connected to the main part of the sleeve near the forward end 38 thereof. For each finger, there is a pair of flexible connections (not shown) between opposed edges of the finger 37 and the adjacent edges of the respective aperture 36. Each finger may thus flex about an axis within the connections. Moreover, each finger is resiliently deformable along its length so that it may serve as a leaf spring by the radially deformation thereof. Though two such apertures 36 each having a respective finger 37 are shown, other numbers of apertures and fingers could be employed, ranging from a single aperture and finger up to three or four apertures and fingers or perhaps even more.

The opposed lugs 30 formed on the mount 28 locate in the apertures 36 of the sleeve and serve to prevent removal of the sleeve 34 from the mount, once fitted thereon and with the sleeve in its initial position shown in FIG. 3. The lugs 30 also serve to limit rotational movement of the sleeve with respect to the mount 28. Each lug 30 has a generally conical forwardly directed surface 39 so as to facilitate fitting of the sleeve to the mount and also to provide a sliding surface for the associated finger, when the sleeve slides with respect to the mount. The abutment surface 31 is formed at the forward end of that surface 39.

A generally ring-shaped control member 40 is disposed within the sleeve 34, that member having a cylindrical forward projection 41 and in the initial setting of the device (FIGS. 1 and 3), the control member is disposed close to the front wall 32 of the mount 28. The external periphery of the control member has a pair of diametrically opposed recessed flanks 42 aligned with the fingers. The control member has a sufficiently large bore 43 to accommodate the main part of a soft needle cover (not shown) and which projects forwardly out of the front end 44 of the sleeve 34, to allow the cover to be gripped manually for removal from the device. The needle cover forms no part of this invention and will not be described in further detail here.

Internally, the rearward end 46 of each finger 37 has a surface for sliding on the ramp surface 29 of the mount 28, as the sleeve 34 slides rearwardly in the course of the performance of an injection procedure. The end face of the rearward end 46 of each finger is formed for engagement with the abutment surface 31 formed at the front of each lug 30 of the mount 28, when the finger is radially aligned with the abutment surface. As shown in the drawings, the free end of each finger is raked slightly and the abutment surface is correspondingly angled such that when the rearward end 46 of the finger engages the abutment surface 31, the finger is encouraged to move deeper into engagement. A small amount of free play may be provided between the rearward end of each finger and the abutment surface, so permitting a limited amount of free movement of the sleeve 34 in its shielding position.

An internal rib 47 is formed at the forward end of each finger, for engagement by the forward projection 41 of the control member 40 in the course of use of the device, as will be described below.

In this embodiment, each of the mount, sleeve and control member is made of a moulded polymeric material. The fingers 37 are resiliently deformable radially outwardly by flexing of those fingers but in the initial position of the device shown in FIGS. 1 and 3, the fingers are undeformed, as are the flexible connections attaching the fingers to the sleeve. As such, the fingers and flexible connections are in their as-moulded unstressed condition. The device may be stored in that condition without the fingers suffering from a loss of resilience, which otherwise would occur through storage if the fingers were deformed, unless made of a material which does not lose resilience if stressed for an extended period.

The operation of the safety device 20 described above will now be explained, following the fitting of the device to a syringe 21. The initial setting (and also the storage position) of the device is shown in FIGS. 1 and 3, with the sleeve 34 in its forward needle-shielding position so as to confer protection to the needle 25. In this setting, the fingers 37 are inclined to the axis of the needle, sleeve and mount and are in an undeformed condition, as mentioned above, so that the bevelled surface may move on to and then slide on the ramp surface 29 of the mount 28, as the sleeve moves rearwardly with respect to the mount 28. The control member 40 is at its set position adjacent the mount 28, allowing the sleeve to move rearwardly.

When an injection is to be performed, the safety device is prepared by grasping and pulling the needle cover out of the device. An injection is performed by offering the forward end 38 of the sleeve 34 to the surface of the skin at an injection site. The syringe 21 is then pushed forwardly while the sleeve remains stationary bearing against the injection site. There is thus relative movement between the sleeve 34 and the syringe 21 and mount 28, the fingers 37 sliding along the lugs 30 during this action. Initially, as shown in FIG. 4, the fingers will in effect turn outwardly about the flexible connections connecting the fingers to the sleeve until contact is established between the control member 40 and the fingers—in the relative positions shown in FIG. 4, there is no contact as the fingers are located in the flanks 42 of the control member, as best seen in FIG. 4A. Continued movement of the syringe 21 forwardly with respect to the sleeve 34 brings the control member to the forward end of the sleeve so that the forward projection 41 of the control member is pushed between the internal ribs 47 at the front of the fingers. This action then generates a turning moment on the fingers in the opposite sense and thus resiliently deforms the parts of the fingers rearwardly of their flexible connections, as shown in FIG. 5. This radially outward deformation of the fingers increases the energy stored within the fingers. In this FIG. 5 position, the needle is projecting fully forwardly from the device.

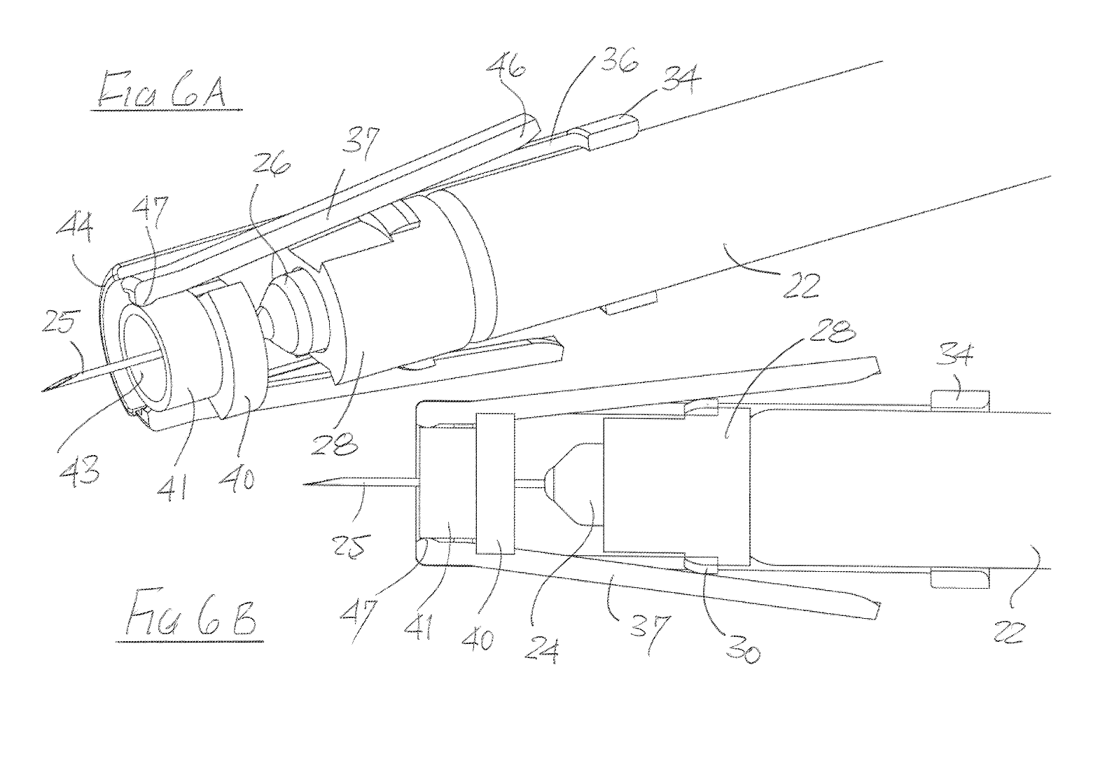

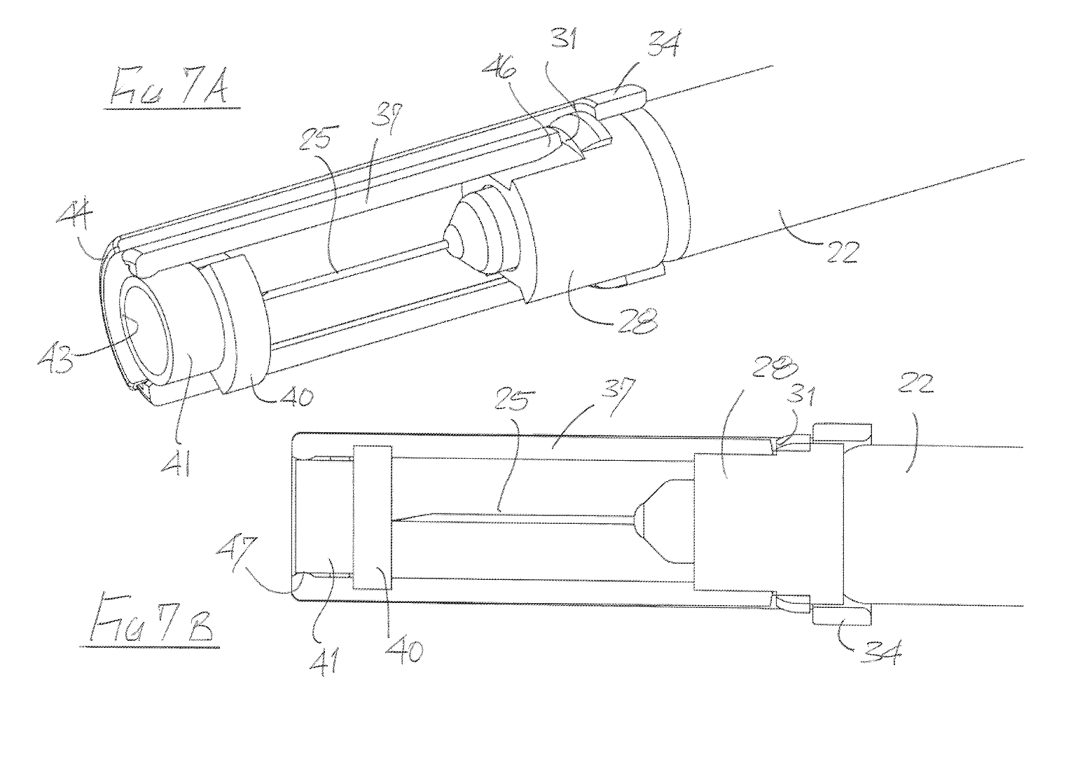

The actual drug injection is then performed by depressing the plunger 23 of the syringe, to dispense the liquid drug through the needle. Once the injection has been completed, the syringe is moved away from the injection site, so withdrawing the needle therefrom. The control member remains at the front end 44 of the sleeve 34 by virtue of the frictional contact between the internal ribs 47 of the fingers with the forward projection 41 of the control member, so maintaining an amount of stored energy in the fingers. Initially, the front end 44 of the sleeve 34 stays in contact with the injection site by virtue of the fingers 37 releasing their stored energy, which in effect moves the sleeve forwardly with respect to the mount 28 and syringe 21 (FIG. 6). When the needle leaves the injection site, the sleeve is moved fully forwardly with respect to the syringe by the restorative force exerted by the fingers 37, increased by the action of the control member on the forward ends of the fingers. The fingers return to their undeformed condition but now in radial alignment with the abutment surface of the sleeve, as shown in FIG. 7. The fingers are turned about the flexible connections to that position by the forward projection 41 of the control member 40 acting on the internal ribs 47 of the fingers. With the radial alignment of the rearward ends 46 of the fingers with the abutment surfaces 31 of the lugs 30, the fingers block subsequent rearward movement of the sleeve. The final shielding position of the sleeve relative to the syringe and mount is substantially the same as the starting position of the sleeve, but the sleeve cannot move rearwardly as such movement is blocked by the fingers being radially aligned with the abutment surfaces.

FIGS. 8 to 10 show the second embodiment of safety device which is generally similar to that described above and like parts are given like reference characters; those parts will not be described again in relation to this second embodiment. The second embodiment differs from the first in that the set position for the control member 50 is at the front end 44 of the sleeve 34 rather than adjacent the mount 28, as in the first embodiment. Consequent upon this, the control member 50 is of a different form, as can be seen in the drawings, though all of the other components of the safety device are as has been described above.

The control member 50 is of a simple cylindrical form having a bore 43 as with the first embodiment, though there is an annular groove 51 formed around the periphery of the control member, adjacent the rear end 52 thereof. The initial position of the control member 50 is shown in FIG. 8, disposed at the front end 44 of the sleeve 34 with the internal ribs 47 of the fingers 37 located in the groove 51. The fingers and the flexible connections attaching the fingers to the sleeve are thus in their as-moulded condition and are essentially unstressed, as with the first embodiment. In this initial set position of the control member, part of the control member projects beyond the front end 44 of the sleeve 34.

As before, the syringe is offered to an injection site but in this case, the projecting part of the control member 50 engages the skin at the injection site. On moving the syringe forwardly to cause the needle 25 to penetrate the injection site, the sleeve initially moves rearwardly with respect to the mount to bring the rear ends 46 of the fingers on to the ramp surfaces 29, after which continued rearward movement of the sleeve starts to deflect the fingers outwardly. As the deflection of the fingers increases, the control member 50 moves rearwardly within the sleeve 34 until the front end 44 of the sleeve engages the injection site, as shown in FIG. 9. In the course of this, the internal ribs 47 of the fingers move out of the groove 51 to bear on the outer cylindrical surface of the control member 50, thus applying a turning moment to the fingers and increasing the deformation thereof. From here, the safety device operates exactly as has been described above with reference to the first embodiment.

At the completion of the injection procedure (FIG. 10), the sleeve 34 is blocked against subsequent rearward movement by the rearward ends 46 of the fingers coming into radial alignment with the abutment surfaces 31. This is assured by the action of the control member on the forward ends of the fingers, applying a turning moment to the fingers about their flexible connection to the sleeve.

FIGS. 11 to 13 show a third embodiment of this invention, differing from the first embodiment in that a simple cylindrical control member 54 is employed and in that as moulded, the fingers of the sleeve are more or less radially aligned with the sleeve. The fingers themselves differ from those of the first and second embodiments in that each finger has an internal cam 55 formed partway therealong but near to the forward ends of the fingers.

The initial position of the control member 54 is as shown in FIG. 11, engaging the cams 55 of the fingers so as to apply a turning moment to the fingers and move the rearward ends 46 thereof outwardly from their undeformed condition into radial alignment with the ramp surfaces 29. The fingers are thus similarly positioned to the fingers of the first embodiment. The rearward ends of the fingers are radially aligned for sliding along the ramp surfaces 29 of the mount 28, readying the device for use.

As with the first embodiment, the front end 44 of the sleeve 34 is offered to skin at an injection site and the syringe 21 is moved forwardly so that the needle 25 penetrates that site. Relative to the syringe, the sleeve moves rearwardly until full penetration of the needle is achieved, as shown in FIG. 12. During the last stage of the forward movement of the syringe, the control member 54 is pushed forwardly relative to the sleeve by the front wall 32 of the mount 28 engaging the rear of the control member 54. This moves the control member out of alignment with the cams 55 and brings the control member into engagement with the internal ribs 47 at the front ends of the fingers, forwardly of their flexible connections. As described above, this applies a turning moment to the fingers, increasing the deformation thereof and so increasing the stored energy in the fingers.

Following the completion of the injection, the syringe is pulled away from the injection site and as has been described with reference to the first and second embodiments, the stored energy of the fingers pushes the sleeve forwardly with respect to the mount and syringe. During this action, by virtue of the friction between the internal ribs 47 of the fingers and the control member 54, the control member remains at the front end of the sleeve. FIG. 13 shows the safety device when the syringe has been moved completely away from the injection site. As before, the rearward ends 46 of the fingers have moved into radial alignment with the abutment surfaces 31 of the lugs 30, so as to block subsequent rearward movement of the sleeve, this action being assured by the action of the control member on the forward ends of the fingers.

The fourth embodiment of this invention is shown in FIGS. 14 to 16 and is generally similar to the third embodiment of FIGS. 11 to 13, except that a different control member 57 is employed. The other components, including the sleeve together with its fingers, are as described in relation to the third embodiment. Thus, as moulded, the fingers of the sleeve are in radial alignment with the ramp surfaces 29, unlike the arrangement of the first two embodiments.

In this fourth embodiment, the control member 57 is somewhat longer than the control member 54 of the third embodiment and between the rear end region 58 and the fore end region 59, there is formed a peripheral recess 60. The set position of the control member is as shown in FIG. 14; here, the fore end region 59 projects beyond the front end 44 of the sleeve and the internal ribs 47 of the fingers are located within the peripheral recess 60. The cams 55 of the fingers are disposed on the rear end region 58 and so the fingers are thrust outwardly to their positions shown in FIG. 14.

An injection is performed by offering the fore end region 59 of the control member 57 to skin at an injection site and then pushing the syringe forwardly so as to have the needle 25 penetrate that site. This has the effect of moving the sleeve 34 rearwardly with respect to the syringe 21 and the mount 28 but after an initial sleeve movement, also moving the control member 57 into the sleeve so that the front of the control member is more or less aligned with the front end 44 of the sleeve as shown in FIG. 15. This brings the internal ribs 47 at the front of the fingers on to the fore end region 59 of the control member 57, so moving the forward ends of the fingers into radial alignment with the sleeve, and applying a turning moment to the fingers. This increases the deformation of the fingers and so also the energy stored in the fingers. From here, the safety device operates exactly as has been described above with reference to the third embodiment of FIGS. 11 to 13. FIG. 16 shows the safety device when the syringe has been moved completely away from the injection site. As before, the rearward ends 46 of the fingers have moved into radial alignment with the abutment surfaces 31 of the lugs 30, so as to block rearward movement of the sleeve for a second time, this action being assured by the action of the control member on the forward ends of the fingers.

The fifth embodiment shown in FIGS. 17 and 18 differs from the previous embodiments in that the sleeve 62 has a smaller diameter than the mount 63 and slides internally within that mount, and the fingers are provided on the mount rather than on the sleeve. The mount is attached to the syringe (not shown) so as to wholly enclose the syringe barrel, though the plunger 64 of the syringe projects rearwardly beyond the mount.

The mount 63 has a par of diametrically opposed elongate apertures 67 within which are provided the fingers 68, each finger being connected to the mount by a flexible connection spaced from but adjacent the rear end of the mount. Each finger and its connection to the mount is similarly formed to the fingers and the connection thereof to the sleeve of the previous embodiments. Thus, at the rear end of each finger 68 there is formed an internally projecting rib 69, for co-operation with a control member 70 forming a part of the plunger 64, to be described below.

The sleeve 62 has a ramp surface 72 at its rear end, for co-operation with the fingers 68 in order to deflect the fingers outwardly as the sleeve slides rearwardly with respect to the mount 63, in the course of performing an injection. The rearwardly directed face of the ramp surface 72 forms the abutment surface of the previous embodiment, for co-operation with the forward ends of the fingers when in radial alignment with the abutment surface, to block relative sliding movement of the sleeve with respect to the mount. As with the previous embodiments, the abutment surface of the sleeve and the forward ends of the fingers are raked slightly such that when the forward end of a finger engages the abutment surface, the finger is encouraged to move deeper into engagement.

As shown in FIG. 17, the control member 70 is moulded integrally with the plunger at the rear end thereof. The control member is circular with a chamfered front edge 73 and is adapted to co-operate with the ribs 69 formed internally at the rearward ends of the fingers 68. As moulded, the fingers initially lie substantially parallel the axis of the mount but with the forward ends of the fingers ready to move on to the ramp surface 72 of the sleeve 62, when the sleeve moves rearwardly with respect to the mount.

FIG. 17 shows the first stage of the performance of an injection procedure, where the plunger has not moved significantly with respect to the syringe barrel but the sleeve 62 has moved rearwardly relative to the mount 63 and so also relative to the syringe. In practice, the mount is grasped by the fingers of a user and the forward end of the sleeve is offered to the skin at an injection site. On pushing the mount forwardly, the mount together with the syringe move forwardly so that the needle penetrates the injection site while the sleeve remains stationary engaged with the skin. As shown, the sleeve has deflected the fingers outwardly and in view of the flexible connection thereof to the mount, the rearward ends of the fingers have moved radially inwardly.

When the sleeve has moved rearwardly to its fullest extent, with the needle then projecting fully into the injection site, the plunger 64 is depressed so as to dispense liquid drug from the syringe. On the control member reaching the rearward ends of the fingers 68, the control member moves between the ribs 69 on the fingers, so moving those rearward ends outwardly more or less into alignment with the mount axis (FIG. 19). This increases the deflection of the fingers and stores further energy therein.

Once the drug has been dispensed by pushing the plunger into the syringe to its fullest extent, the mount is moved away from the injection site. This allows the stored energy of the fingers 68 to move the sleeve 62 forwardly relative to the mount until the forward end of the sleeve comes clear of the injection site. The sleeve continues to move forwardly under the action of the fingers which, on account of the control member remaining between the ribs 69 on the fingers, ensures that the fingers have sufficient stored energy fully to move the sleeve to its initial position, and the forward ends of the fingers move radially inwardly beyond the initial as-moulded condition so that the forward ends thereof become radially aligned with the abutment surface of the sleeve. As with the previous embodiments, this then blocks the sleeve against rearward movement for a second time.

Though in the fifth embodiment described above the control member 70 is moulded integrally with the plunger 64, the control member could be separately formed and then attached to the plunger at a suitable position having a regard to the required stroke of the plunger. Another possibility would be to allow the control member to be free to slide on the plunger. For such an arrangement, the control member could have a peripheral groove formed adjacent the forward end thereof, with the ribs 69 on the fingers located in that groove at the initial setting of the device. The control member would remain stationary with respect to the fingers until the plunger has almost reached its final position. Then the plunger will move the control member forwardly such that the ribs 69 will be moved out of the groove and onto the larger diameter part of the control member. Such an arrangement is similar to the second embodiment described above.

1. A safety device for shielding a medical needle having a sharp tip, which device comprises:
a needle mount for directly or indirectly supporting a medical needle;
a needle shielding sleeve for co-axially surrounding a supported needle and arranged for sliding movement from an initial needle shielding position to a non-shielding position whereat at least the tip of a supported needle is exposed beyond the sleeve;
an abutment surface and a sliding surface provided on one of the sleeve and mount;
a radially deformable resilient finger provided on the other of the sleeve and mount by a flexible connection, a first part of the finger to one side of the connection being configured for radial alignment with the abutment surface or the sliding surface whereby when in radial alignment with the abutment surface movement of the sleeve from its initial needle shielding position is blocked and when in radial alignment with the sliding surface movement of the sleeve from its initial shielding position is permitted while causing deformation of the finger thereby generating a restorative force on the sleeve to urge the sleeve back to a shielding position; and
a control member arranged for coastal movement with respect to the linger and co-operable with a second part thereof to the other side of said flexible connection, the control member having a set position with respect to the finger at which said first part of the finger is in radial alignment with the sliding surface so allowing movement of the sleeve from its initial shielding position, the control member also having an active position displaced from set position and whereat said second part of the finger is moved outwardly so urging the first part of the finger inwardly into radial alignment with the abutment surface following the return of the sleeve to a shielding position thereby to block subsequent movement of the sleeve away therefrom, the control member being moved to its active position by the use of the device.

2. A safety device for shielding a medical needle having a sharp tip, which device comprises:
a needle mount for directly or indirectly supporting a medical needle;
a needle shielding sleeve for surrounding the needle and arranged coaxially with the mount so that a force applied to the sleeve slides the sleeve from an initial needle shielding position to a non-shielding position whereat at least the tip of the needle is exposed beyond the sleeve;
an abutment surface and a sliding surface provided oh one of the sleeve and mount;
a radially deformable resilient finger provided on the other of the sleeve and mount by a flexible connection, a first part of the finger to one side of the connection being configured for radial alignment with the abutment surface or the sliding surface and when in radial alignment with the abutment, surface the finger blocks movement of the sleeve from its initial, needle shielding position; and
a control member slidably arranged coaxially with the sleeve and mount and co-operable with a second part of the finger to the other side of said flexible connection, the control member having a set position with respect to the sleeve at which said first part of the linger is in radial alignment with the sliding surface so allowing movement of the sleeve from its initial shielding position, and movement of the sleeve towards its non-shielding position causes deformation of the finger thereby generating a restorative force between the finger and the sliding surface that urges the sleeve back to a shielding position, and said movement of the sleeve from the initial shielding position causes the control member to be displaced from the set position;
wherein, the movement, of the control member from its set position and the return of the sleeve to a shielding position moves said second part of the finger outwardly and by virtue of the flexible connection of the finger also urges the first part of the finger inwardly and so into radial alignment with the abutment surface following the return of the sleeve to a shielding position, to block subsequent movement of the sleeve away therefrom.

3. A safety device as claimed in claim 1, wherein the control member is tubular and surrounds the needle, the control member being arranged for axial sliding movement from its set position.

4. A safety device as claimed in claim 1, wherein the mount is of a smaller diameter than the sleeve such that the sleeve slides over the mount, and the finger is carried by the sleeve to project rearwardly for contacting the abutment surface or the sliding surface provided on the mount.

5. A safety device as claimed in claim 4, wherein the hinging connection of the finger to the sleeve is disposed near to but spaced back from the forward end of the sleeve.

6. A safety device as claimed in claim 1, wherein the control member when in its set position is disposed adjacent the mount and is arranged to slide therefrom towards the forward end of the sleeve as the sleeve slides rearwardly from its initial shielding position to expose at least the tip of the needle as aforesaid.

7. A safety device as claimed in claim 1, wherein said one part of the finger is located at the rearward end thereof, and said second part of the finger is located at the forward end thereof.

8. A safety device as claimed in claim 1, wherein the control member when in its set position is disposed at the forward end of the sleeve and projects therebeyond, the control member being arranged to slide rearwardly from the set position and act on the second part of the finger at the forward end of the sleeve as the sleeve slides rearwardly from its initial shielding position to expose at least the tip of the needle as aforesaid.

9. A safety device as claimed in claim 1, wherein the mount is adapted to move the control member from its set position on sliding movement of the sleeve away from its initial shielding position.

10. A safety device as claimed in claim 1, wherein the control member when in its set position is disposed at the forward end of the sleeve and projects therebeyond, the control member having a region which co-acts with the first part of the finger to stress the connection and move said one part into radial alignment with the sliding surface, the control member being arranged to slide rearwardly from the set position and act on the second part of the finger at the forward end of the sleeve as the sleeve slides rearwardly from its initial shielding position to expose at least the tip of the needle as aforesaid.

11. A safety device as claimed in claim 10, wherein the control member has a recess to accommodate the forward end of the finger when the control member is in its set position, arid the forward end of the finger comes out of the recess when the control member moves rearwardly away from its set position.

12. A safety device as claimed in claim 1, wherein the control member is mouthed remotely of the sleeve for movement with respect to the mount.

13. A safety device as claimed in claim 1 in combination with an injection device comprising a syringe having a medical needle with a sharp tip, a plunger for expelling a contained liquid drug though the needle and a safely device for shielding the needle, wherein the control member is associated with the plunger and is arranged such that movement of the plunger to expel the drug from the syringe displaces the control member from the set position and moves said second part of the finger outwardly so urging the first part of the finger inwardly by virtue of the flexible connection of the finger.

14. A safety device as claimed in claim 13, wherein the mount is of a larger diameter than the sleeve such that the sleeve slides within the mount, the abutment surface and the sliding surface are provided on the sleeve and the finger is carried by the mount to project forwardly for contacting the abutment surface or the sliding surface on the sleeve.

15. A safety device as claimed in claim 13, wherein, the mount substantially contains the syringe and the control member is provided on a plunger associated with the syringe, to co-operate with the rearward end of the finger,

16. A safety device as claimed in claim 1, wherein the hinging connection allows resilient hinging movement of the finger and when the finger is not deformed and has said one part radially aligned with the sliding surface, the hinging connection is in an unstressed condition.

17. A safety device as claimed in claim 1, wherein the control member has a recess to accommodate a portion of the second part of the finger when the control member is in its set position, and said portion comes out of the recess when the control member is moved from its set position.

18. A safety device as claimed in claim 1, wherein the hinging connection allows resilient hinging movement of the finger and when the control member is in its set position, the control member acts on the finger to stress the connection and move said one part into radial alignment with the sliding surface.

19. A safety device as claimed in claim 18, wherein the set position of the control member is with the control member acting on said one part of the finger adjacent the hinging connection, and the control member moves from its set position to act on the second part of the finger, to the other side of the hinging connection.

20. A safety device as claimed in claim 1, wherein the needle mount has a bore for receiving a hub of a medical needle from which a needle projects forwardly.

21. A safety device as claimed in claim 20, wherein the hub of the medical needle comprises a part of a syringe having a needle permanently affixed thereto and projecting forwardly therefrom, the mount being adapted for connection to the hub.

22. A safety device as claimed in claim 1, wherein the sliding surface of said other of the sleeve and the mount is defined by an upstanding lug and the finger has a radially inner lace which slides thereon.

23. A safety device as claimed in claim 22, wherein said abutment surface comprises a surface formed as apart of the log.

24. A safety device as claimed in claim 2, wherein the control member is slidably carried within the larger diameter of the sleeve and mount, and is held against rotation with respect thereto.

25. A safety needle assembly comprising a safety device as claimed in claim 1 in combination with a medical needle housed within the device and shielded by the sleeve when in its shielding position.

26. An injection device comprising a: safety needle assembly as claimed in claim 24 in combination with a syringe or injector arranged to co-operate with the assembly to permit the performance of a medical procedure with the medical needle but imparting passive safety on the sharp tip of the needle.

27. A safety device as claimed in claim 2, wherein the control member is tubular and surrounds the needle, the control member being arranged for axial sliding movement from its set position.

28. A safety device as claimed in claim 27, wherein the mount is of a smaller diameter than the sleeve such that the sleeve slides over the mount, and the linger is carried by the sleeve to project rearwardly for contacting the abutment surface or the sliding surface provided on the mount,

29. A safety device as claimed in claim 2, wherein said one part of the finger is located at the rearward end thereof and said second part of the finger is located at the forward end thereof.

30. A safely device as claimed in claim 2, wherein the control member when in its set position is disposed at the forward end of the sleeve and projects therebeyond, the control member being arranged to slide rearwardly from the set position and act on the second part of the finger at the forward end of the sleeve as the sleeve slides rearwardly from its initial shielding position to expose at least the tip of the needle as aforesaid.
